(19) United States
(12) Patent Application Publication
(10) Pub. No.: US 2025/0229064 A1
(43) Pub. Date: Jul. 17, 2025

(54) NASOJEJUNAL FEEDING TUBE WITH BALLOON FOR RETENTION

(71) Applicant: BOSTON SCIENTIFIC SCIMED, INC., Maple Grove, MN (US)

(72) Inventors: Peter L. DAYTON, Brookline, MA (US); John Thomas FAVREAU, Charlton, MA (US); John B. GOLDEN, Norton, MA (US); Kurt Nicholas ROBAKIEWICZ, Upton, MA (US); Laura Elizabeth CHRISTAKIS, Framingham, MA (US)

(21) Appl. No.: 19/016,150

(22) Filed: Jan. 10, 2025

Related U.S. Application Data
(60) Provisional application No. 63/621,864, filed on Jan. 17, 2024.

(51) Int. Cl. A61M 25/10; A61M 25/00

(52) U.S. Cl. CPC A61M 25/1011; A61M 2025/0006; A61M 2025/1015; A61M 2025/1086; A61M 2202/0007; A61M 2202/04; A61M 2205/0216; A61M 2210/1042

(57) ABSTRACT

A device includes a balloon and a catheter having a first channel. The catheter is configured to extend from a proximal end outside a body to a distal end so that it can be guided to a location within a GI system. A first distal portion of the catheter includes exit port(s) communicating with a second channel for administering a fluid to the location. The catheter includes a third channel communicating with an interior of the balloon so that, in a first state, the balloon is deflated and assumes a first shape for guiding the catheter to the location and, in a second state, the balloon is inflated and assumes a second shape in which the balloon extends radially from the catheter for contacting a wall of the location to anchor the catheter in the location while administering the fluid via the first channel.

### PRIORITY CLAIM

The present disclosure claims priority to U.S. Provisional Patent Application Ser. No. 63/621,864 filed Jan. 17, 2024; the disclosure of which is incorporated herewith by reference.

### FIELD

The present disclosure relates to tubes and/or catheters with retention features for anchoring in a target organ such as the small bowel.

### BACKGROUND

Pyloric stenosis constitutes a narrowing of the gastric outlet (pylorus) causing difficulties in passing food from the stomach into the small bowel. This can lead to a gastric outlet obstruction (stricture). The inability to pass food into the small bowel can reduce or completely impede a person's ability to digest food and absorb nutrients. There are several solutions to this problem, including placing a nasojejunal (NJ) feeding tube in the small bowel, e.g., jejunum. Another solution includes the creation of a surgical gastro-jejunostomy bypass bridging the stomach and the jejunum to bypass the area of the stricture. NJ tubes can be used for assisting endoscopic ultrasound guided gastroenterostomy (EUS-GE) procedures by instilling contrast or another fluid in the jejunum. In one example, contrast is provided via the NJ tube to help make visible a target to an operating physician, e.g., for a procedure including piercing the stomach and the jejunum and placing a stent (e.g., AXIOS stent) therebetween to bypass the pylorus.

One issue with NJ tubes is that the tube may drift out of position during use so that fluids discharged by the tube are provided to an unintended location (i.e., as the outlet of the tube is no longer located in a target location). In one example, the NJ tube may migrate proximally during use. In feeding tube applications, this proximal drift may cause nutrients to be provided further upstream in the small bowel, or in the stomach proximal to the stricture, which would eliminate the benefits of the tube and may cause issues with digestion. In EUS-GE applications, the drift of the NJ tube can cause fluids (e.g., contrast fluid) to be provided to a location spaced from an intended target area. The stents for bypassing the pylorus may be mis-deployed if the physician is confused or misdirected due to the contrast fluid being provided to an unintended location during gastro-jejunostomy procedures.

### SUMMARY

The present disclosure relates to a device for administering a fluid to a patient anatomy. The device includes a catheter including a first channel extending therethrough for passing the catheter over a guidewire, the catheter being sized and shaped to extend from a proximal end outside a human body to a distal end that can be guided to a target location within a gastrointestinal (GI) system, a first distal portion of the catheter including one or more exit ports communicating with a second channel for administering a fluid to the target location. In addition, the device includes a balloon distal to the exit ports, the catheter including a third channel extending therethrough communicating with an interior of the balloon so that, in a first state, the balloon is deflated and assumes a first shape suitable for guiding the catheter to the target location and, in a second state, the balloon is inflated and assumes a second shape in which the balloon extends radially from the catheter for contacting a wall of the target location to anchor the catheter in the target location while administering the fluid via the first channel.

In an embodiment, the fluid administered to the target location comprises contrast and the balloon prevents the fluid from migrating distally.

In an embodiment, the balloon comprises a micropatterned surface for adhering to the wall of the target location.

In addition, the present disclosure relates to a device for administering a fluid to a patient anatomy. The device includes a first catheter that is an outer catheter including a first channel extending therethrough, the first catheter being sized and shaped to extend from a proximal end outside a human body to a distal end that can be guided to a first target location within a gastrointestinal (GI) system. In addition, the device includes a first balloon adjacent to the distal end of the first catheter, the first catheter including a second channel extending therethrough communicating with an interior of the first balloon so that, in a first state, the first balloon is deflated and assumes a first shape suitable for guiding the first catheter to the first target location and, in a second state, the first balloon is inflated and assumes a second shape in which the first balloon extends radially from the first catheter for contacting a wall of the first target location to anchor the first catheter in the first target location.

Furthermore, the device includes a second catheter that is an inner catheter sized and shaped to be received within the first channel of the first catheter, the second catheter extending from a proximal end outside the human body through the first channel to a distal end that that, in a first position, is distal to the distal end of the first catheter, the second catheter being translatable relative to the first catheter such that, after the first balloon has anchored the first catheter in the first target location, the second catheter can be guided to a second target location within the GI system. In addition, the device includes a second balloon adjacent to the distal end of the second catheter, the second catheter including a third channel extending therethrough communicating with an interior of the second balloon so that, in a first state, the second balloon is deflated and assumes a first shape suitable for guiding the second catheter to the second target location and, in a second state, the second balloon is inflated and assumes a second shape in which the second balloon extends radially from the second catheter for contacting a wall of the second target location to anchor the second catheter in the second target location.

In an embodiment, the device includes further comprising a ratcheting mechanism wherein a medial portion of the second catheter comprises ridges along a length of the medial portion, wherein the distal end of the first catheter comprises a protrusion extending radially inward to engage a space between adjacent ridges to maintain a position of the second catheter relative to the first catheter, the protrusion being flexible so that an axial force applied to the second catheter deforms the protrusion allowing the second catheter to translate so that the protrusion engages a further space between further adjacent ridges on the medial portion.

In an embodiment, either the first catheter or the second catheter includes a fourth channel for administering a fluid to a volume between the first and second target locations, the volume being distal to the first balloon and proximal to the second balloon.

In an embodiment, either one or both of the first balloon and the second balloon comprises a micropatterned surface for adhering to the wall of the first target location or the second target location.

In addition, the present disclosure relates to a device for administering a fluid to a patient anatomy. The device includes a catheter sized and shaped to extend from a proximal end outside a human body to a distal end that can be guided to a target location within a gastrointestinal (GI) system; a first balloon that is an inner balloon adjacent to the distal end of the catheter, the catheter including a first channel extending therethrough communicating with an interior of the first balloon so that, in a first state, the first balloon is deflated and assumes a first shape suitable for guiding the catheter to the target location and, in a second state, the first balloon is inflated with a fluid having properties to enhance visibility for a fluoroscopic or endoscopic procedure; and a second balloon that is an outer balloon disposed around the first balloon, the catheter including a second channel extending therethrough communicating with an interior of the second balloon so that, in a first state, the second balloon is deflated and assumes a first shape suitable for guiding the catheter to the target location and, in a second state, the second balloon is inflated and assumes a second shape in which the second balloon extends radially from the catheter for contacting a wall of the target location to anchor the catheter in the target location.

In an embodiment, the first balloon provides a target for the fluoroscopic or endoscopic procedure and is formed of a first material stronger than a second material of the second balloon.

In an embodiment, the second balloon is punctured and the first balloon is not punctured during the fluoroscopic or endoscopic procedure.

In an embodiment, the second balloon comprises a micropatterned surface for adhering to the wall of the target location.

In addition, the present disclosure relates to a device for administering a fluid to a patient anatomy. The device a catheter including a first channel extending therethrough, the catheter being sized and shaped to extend from a proximal end outside a human body to a distal end that can be guided to a target location within a gastrointestinal (GI) system, a first distal portion of the catheter including one or more exit ports communicating with the first channel for administering a fluid to the target location; and a balloon distal to the exit ports, the catheter including a second channel extending therethrough communicating with an interior of the balloon so that, in a first state, the balloon is deflated and assumes a first shape suitable for guiding the catheter to the target location and, in a second state, the balloon is inflated and assumes a second shape in which the balloon extends radially from the catheter for contacting a wall of the target location to anchor the catheter in the target location while administering the fluid via the first channel. A single region of the balloon is attached to a single region of the catheter so that the balloon can be displaced proximally and distally to the single region of the catheter under axial forces imposed by the GI system.

In an embodiment, the fluid administered to the target location comprises contrast and the balloon prevents the fluid from migrating distally.

In an embodiment, the balloon comprises ribs for adhering to the wall of the target location.

In an embodiment, the balloon comprises a micropatterned surface for adhering to the wall of the target location.

In addition, the present disclosure relates to a method for administering a fluid to a patient anatomy. The method includes guiding a catheter to a target location within a gastrointestinal (GI) system, the catheter including a first channel extending therethrough for passing the catheter over a guidewire, the catheter being sized and shaped to extend from a proximal end outside a human body to a distal end, a first distal portion of the catheter including one or more exit ports communicating with a second channel for administering a fluid to the target location, the catheter including a third channel extending therethrough communicating with an interior of a balloon distal to the exit ports; transitioning the balloon from a first state to a second state, wherein in the first state the balloon is deflated and assumes a first shape suitable for guiding the catheter to the target location and, in the second state, the balloon is inflated and assumes a second shape in which the balloon extends radially from the catheter for contacting a wall of the target location to anchor the catheter in the target location; and administering the fluid via the first channel.

In an embodiment, the fluid administered to the target location comprises contrast and the balloon prevents the fluid from migrating distally.

In an embodiment, the balloon comprises a micropatterned surface for adhering to the wall of the target location.

In addition, the present disclosure relates to a method for administering a fluid to a patient anatomy including guiding a first catheter to a first target location within a gastrointestinal (GI) system, the first catheter being an outer catheter including a first channel extending therethrough, the first catheter being sized and shaped to extend from a proximal end outside a human body to a distal end, the first catheter including a second channel extending therethrough communicating with an interior of a first balloon adjacent to the distal end of the first catheter; transitioning the first balloon from a first state to a second state, wherein, in the first state, the first balloon is deflated and assumes a first shape suitable for guiding the first catheter to the first target location and, in a second state, the first balloon is inflated and assumes a second shape in which the first balloon extends radially from the first catheter for contacting a wall of the first target location to anchor the first catheter in the first target location; guiding a second catheter to a second target location within the GI system, the second catheter being an inner catheter sized and shaped to be received within the first channel of the first catheter, the second catheter extending from a proximal end outside the human body through the first channel to a distal end that that, in a first position, is distal to the distal end of the first catheter, the second catheter being translatable relative to the first catheter such that, after the first balloon has anchored the first catheter in the first target location, the second catheter can be guided to the second target location, the second catheter including a third channel extending therethrough communicating with an interior of a second balloon adjacent to the distal end of the second catheter; and transitioning the second balloon from a first state to a second state, wherein, in the first state, the second balloon is deflated and assumes a first shape suitable for guiding the second catheter to the second target location and, in a second state, the second balloon is inflated and assumes a second shape in which the second balloon extends radially from the second catheter for contacting a wall of the second target location to anchor the second catheter in the second target location.

In an embodiment, the guiding the second catheter to the second target location utilizes a ratcheting mechanism wherein a medial portion of the second catheter comprises ridges along a length of the medial portion, wherein the distal end of the first catheter comprises a protrusion extending radially inward to engage a space between adjacent ridges to maintain a position of the second catheter relative to the first catheter, the protrusion being flexible so that an axial force applied to the second catheter deforms the protrusion allowing the second catheter to translate so that the protrusion engages a further space between further adjacent ridges on the medial portion.

### BRIEF DESCRIPTION

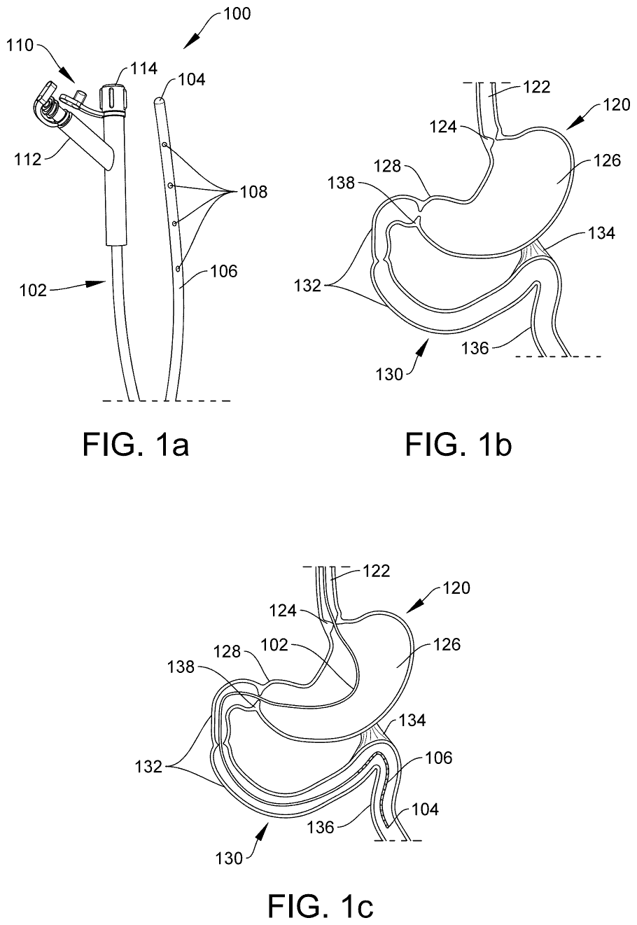

FIG. 1a shows a nasojejunal (NJ) tube device according to one example.

FIG. 1b shows a patient anatomy prior to inserting a NJ tube according to one example.

FIG. 1c shows the catheter of the NJ tube device of FIG. 1a placed in the patient anatomy of FIG. 1b according to one example.

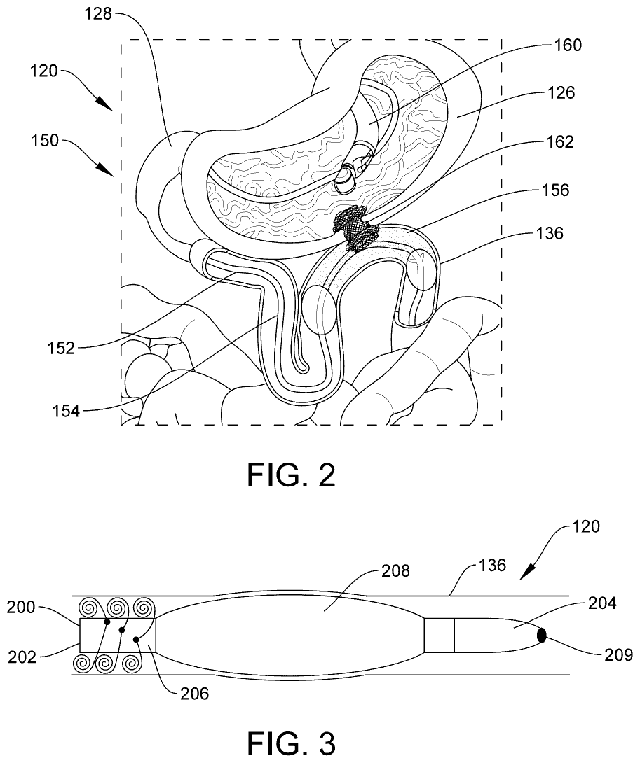

FIG. 2 shows a EUS-GE system for placing a gastro-jejunal stent in the anatomy of FIG. 1b according to one example.

FIG. 3 shows a catheter device including a catheter with a balloon fixed thereto in an expanded shape (second shape) within the patient anatomy of FIG. 1b according to a first example of these exemplary embodiments.

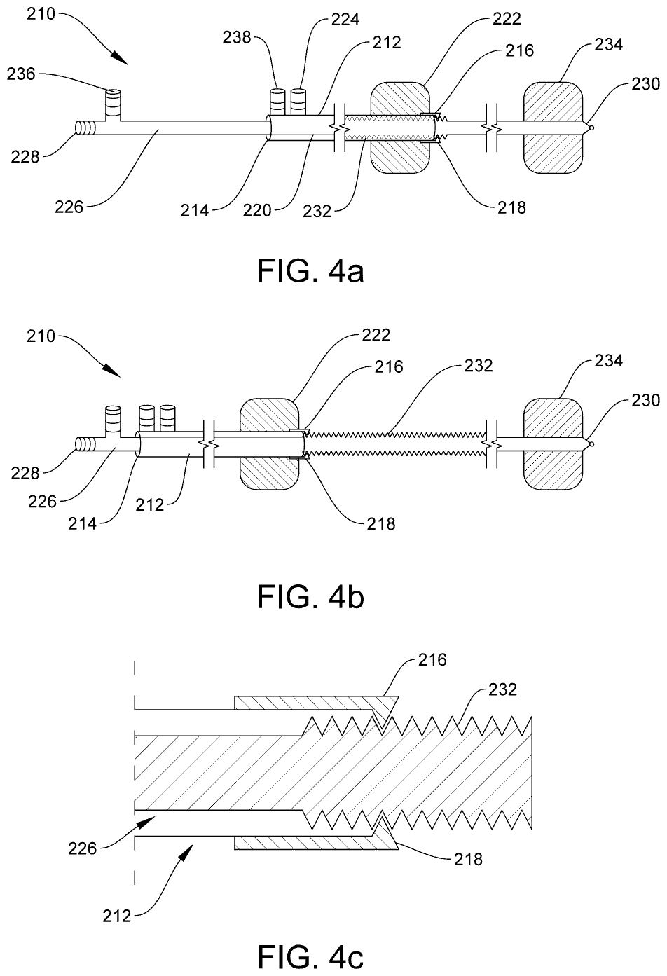

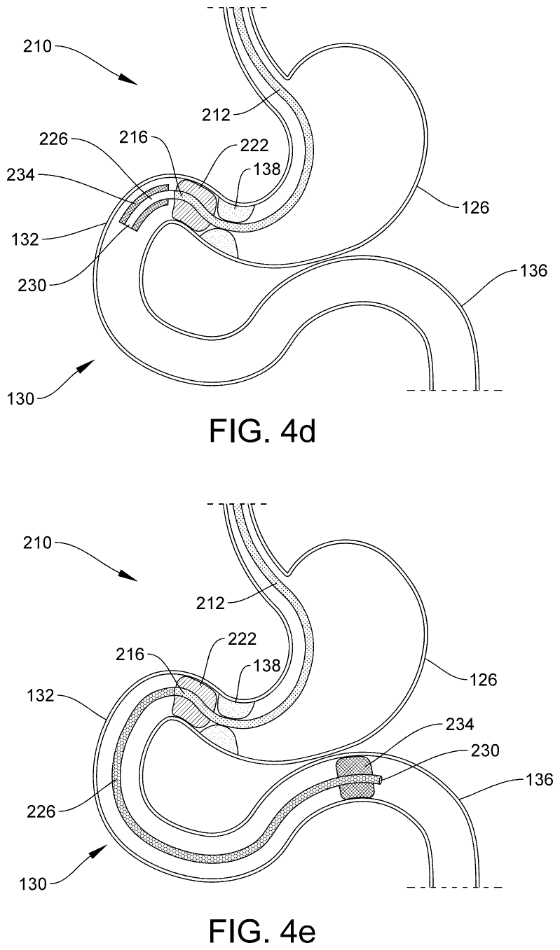

FIG. 4a shows a catheter device including a first catheter (outer catheter) with a first balloon fixed thereto, a second catheter with a second balloon fixed thereto and a ratcheting mechanism for setting a distance between the balloons according to a second example of these exemplary embodiments.

FIG. 4b shows the catheter device of FIG. 4a with the second catheter advanced distally relative to the first catheter according to the second exemplary embodiment.

FIG. 4c shows the ratcheting mechanism of the catheter device of FIG. 4a according to the second exemplary embodiment.

FIG. 4d shows the catheter device of FIG. 4a within the patient anatomy of FIG. 1b in a first stage of deployment according to the second exemplary embodiment.

FIG. 4e shows the catheter device of FIG. 4a within the patient anatomy of FIG. 1b in a second stage of deployment according to the second exemplary embodiment.

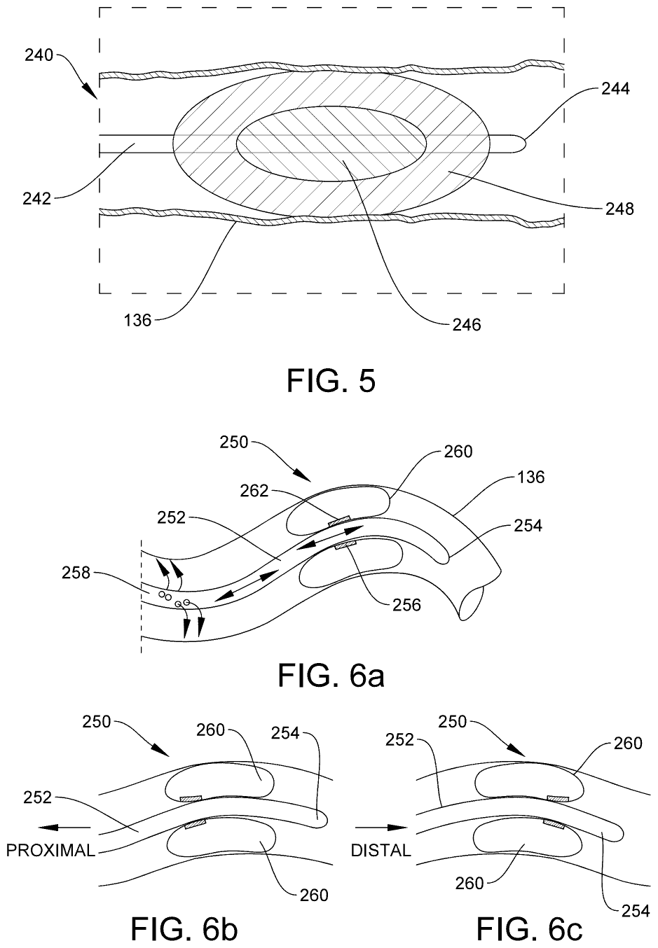

FIG. 5 shows a catheter device including an outer balloon and an inner balloon fixed near a distal end of a catheter within the patient anatomy of FIG. 1b according to a third example of these exemplary embodiments.

FIG. 6a shows a catheter device including a rolling balloon fixed to a catheter within the patient anatomy of FIG. 1b according to a fourth example of these exemplary embodiments.

FIG. 6b shows the balloon of the catheter device of FIG. 6a displaced proximally in the patient anatomy of FIG. 1b according to the fourth exemplary embodiment.

FIG. 6c shows the balloon of the catheter device of FIG. 6a displaced distally in the patient anatomy of FIG. 1b according to the fourth exemplary embodiment.

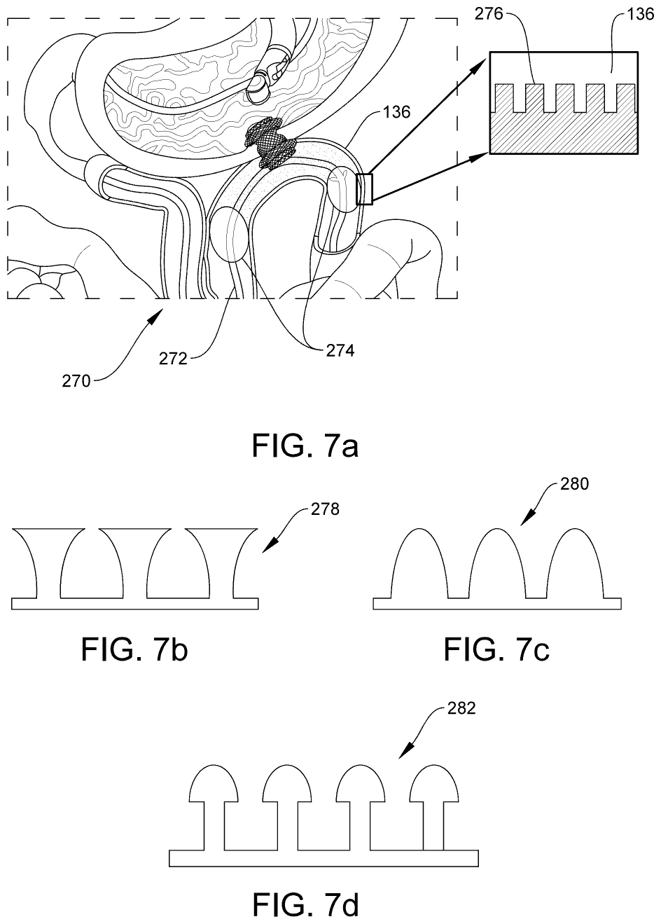

FIG. 7a shows a EUS-GE system comprising a balloon with a first micropatterned surface according to a fifth example of these exemplary embodiments.

FIG. 7b shows a second micropatterned surface according to a sixth example of these exemplary embodiments.

FIG. 7c shows a third micropatterned surface according to a seventh example of these exemplary embodiments.

FIG. 7d shows a fourth micropatterned surface according to an eighth example of these exemplary embodiments.

### DETAILED DESCRIPTION

The present disclosure may be further understood with reference to the following description and the appended drawings, wherein like elements are referred to with the same reference numerals. The present disclosure relates to nasojejunal (NJ) tube and/or catheter devices including features for retention at a target location in the gastrointestinal (GI) tract. In particular, the exemplary catheter devices include at least one balloon coupled to a catheter, the balloon configured to transition from a deflated state (a first shape or state) suitable for advancing the catheter through the GI tract to an expanded state (a second shape or state) in which the balloon expands to engage an inner wall of the small bowel, e.g., the duodenum or the jejunum.

Pyloric stenosis involves a narrowing of the gastric outlet (pylorus) which may cause difficulties in passing food from the stomach to the small bowel. This may lead to gastric outlet obstruction (stricture) reducing or completely impeding the ability of the patient to digest food and absorb nutrients. As indicated above, there are several currently employed treatments for this problem, including placing a nasojejunal (NJ) feeding tube in the small bowel. The NJ tube is fed down the esophagus into the stomach, e.g., via endoscope or guidewire, and guided through the stricture so that nutrients may be provided to the small bowel, e.g., the jejunum, using the tube to bypass the stricture. The NJ tube typically comprises holes along a length of a portion of the tube near the distal end of the tube so that this portion, when positioned as desired, discharges nutrients from the tube into the target anatomical structure (e.g., small bowel) via the holes.

FIG. 1a shows a nasojejunal (NJ) tube device 100 according to one example. The device includes a catheter 102 (e.g., tube) extending from a proximal end (not shown) to a distal end 104 with an atraumatic distal tip configured to be advanced through a portion of the gastrointestinal (GI) tract until the distal end 104 is positioned in a desired location. The catheter 102 of this example comprises a single lumen or channel extending therethrough. The catheter 102 has a distal portion 106 near or adjacent to the distal end 104 comprising holes 108 configured to discharge a fluid administered through the lumen of the catheter 102 into a target anatomical structure. In short, the distal portion 106 of the catheter 102 extends along a portion of the catheter 102 that is configured to be positioned within a target anatomical area into which fluids are to be supplied via the catheter. The catheter 102 of this embodiment is configured (has a length and diameter selected) to be guided through the GI tract to a position in which the distal portion 106 is located in the small bowel, e.g., the jejunum, while the proximal end remains outside the patient's body accessible to a user.

The NJ tube device 100 further comprises a handle 110 at its proximal end including a port 112 in fluid communication with and configured to access the lumen of the catheter 102, e.g., to administer the fluid from the port 112 to the target anatomical structure via the lumen of the catheter 102. The catheter 102 is configured (sized and shaped) to be advanced longitudinally through a channel of the handle 110 as the catheter 102 is guided to the target location. The proximal end of the catheter 102 is fixed to a cap 114 configured to interface with the handle 110 when the catheter 102 is fully extended distally relative to the handle 110.

FIG. 1b shows an exemplary portion of a patient's anatomy 120 prior to inserting a NJ tube according to one example. The anatomy 120 comprises a portion of the GI tract including the esophagus 122, the stomach 126 and the small bowel 130 (small intestine). The small bowel 130 includes the duodenum 132, the jejunum 136, and the ileum (not shown). As would be understood by those skilled in the art, the esophageal sphincter 124 corresponds to the end of the esophagus 122 and the start of the stomach 126, the pylorus 128 corresponds to the end of the stomach 126 and the start of the duodenum 132 and the ligament of Treitz 134 is a ligament supporting the small bowel and corresponds to the end of the duodenum 132 and the start of the jejunum 136. Pyloric stenosis refers to a narrowing of the pylorus 128 that can create a stricture 138 preventing or making difficult the passage of food from the stomach 126 to the small bowel 130.

FIG. 1c shows the catheter 102 of the NJ tube device 100 of FIG. 1a placed in the anatomy 120 of FIG. 1b according to one example. The catheter 102 of the NJ tube device 100 is configured to be passed down the esophagus 122, through the stomach 126, past the stricture 138 (e.g., the pylorus 128 which has narrowed excessively) and into the small bowel 130 under visualization (e.g., via endoscope) by using a guidewire, and/or by other methods. The catheter 102 is configured to be passed through the duodenum 132 past the ligament of Treitz 134 to the jejunum 136 so that the distal portion 106 comprising the holes 108 is positioned at least partially in the jejunum 136. Once the catheter 102 has been placed at the desired location, fluids can be administered via the handle 110 and discharged into the jejunum 136 via the holes 108.

In this example, the NJ tube device 100 is for feeding purposes and the fluids administered by the tube comprise nutrients for the patient. In other examples, the fluid can comprise contrast or other therapeutic fluids, e.g., medicines, fluids configured to enhance visualization for assisting endoscopic ultrasound guided gastroenterostomy (EUS-GE) procedures, etc.

Another method for treating a patient with pyloric stenosis includes the creation of a surgical gastro-jejunostomy bypass bridging the stomach and the jejunum to bypass the area of the stricture. NJ tubes can be used to assist with EUS-GE procedures by instilling contrast or another fluid into the jejunum to enhance visualization of the target anatomy. In one example, contrast is provided to enhance visualization of the target anatomy by an operating physician, e.g., for a procedure including piercing the stomach and the jejunum and placing a stent (e.g., AXIOS stent) therebetween to bypass the pylorus.

FIG. 2 shows an EUS-GE system 150 for placing a gastro-jejunal stent 162 in the anatomy 120 of FIG. 1b according to one example. The EUS-GE system 150 comprises a NJ tube device 152 including a catheter 154 that is passed into the jejunum 136 so that fluid can be discharged into the jejunum 136. In this example, the fluid comprises contrast fluid 156 to help the operating physician locate the target location.

The EUS-GE system 150 of this example further comprises an endoscope 160 for deploying the stent 162. A piercing element (not shown) can be deployed via the endoscope 160 to pierce the stomach 126 and the jejunum 136 as would be understood by those skilled in the art. The stent 162 is placed to bridge the stomach 126 and the jejunum 136 bypassing the pylorus 128.

One issue with certain existing NJ tubes has been a tendency for the tube to drift out of position during use such that fluids discharged by the tube may be provided to an unintended location. In one example, the NJ tube can migrate proximally during use. In feeding tube applications, this proximal drift can cause nutrients to be provided via the feeding tube to a location further upstream in the small bowel than the target location, or even to a location in the stomach proximal to the stricture, which may cause issues with digestion.

In EUS-GE applications, the drift of the NJ tube can cause fluids (e.g., contrast fluid) to be instilled away from an intended target area. With regard to the gastro-jejunostomy procedure described in FIG. 2, without proper visualization, stents for bypassing the pylorus may be mis-deployed. In some examples, the stent may be deployed in a hole created in the stomach but without capturing the jejunum (which may or may not have been pierced by the piercing element); similarly, the stent can be deployed in the hole created in the jejunum but without capturing the stomach; or the stent may be deployed to bridge the stomach and the colon. Accordingly, there is a need for improved devices and mechanisms to retain a tube in a desired location in the GI tract.

According to various exemplary embodiments, systems, devices and methods are described for retaining a catheter, e.g., a nasojejunal tube, at a desired location in a target organ such as the small bowel. The catheter can be retained at a position in the small bowel such that the holes for discharging a fluid are positioned in a desired location, e.g., the jejunum. Some of the devices described herein include a catheter intended for use as a feeding tube, e.g., for extended use (weeks, months, etc.), and some of the devices described herein include catheters intended for providing fluid during EUS-GE procedures, as will be described below. However, it is noted that many of the described embodiments are equally applicable for either purpose, as described in detail below.

The terms “NJ tube” and/or “catheter” are used herein to describe a tube configured for deployment within the GI tract for administering a fluid, e.g., for feeding, a therapeutic fluid or a fluid applied to facilitate a procedure such as EUS-GE. Some catheters according to the present embodiments have a single lumen or channel for passing a fluid through the catheter from a proximal end to a distal portion from which the fluid can be discharged through holes in the wall of the catheter. In other embodiments, the catheter can include multiple lumens for facilitating the actuation of the retention features, to be described below.

Those skilled in the art understand that a simple catheter can comprise a length of tube, e.g., formed of a material such as a plastic and/or polymer (e.g., PVC, PTFE, etc.) with a desired flexibility and/or rigidity, that may assume, for example, a substantially straight (longitudinal) or slightly curved shape when no external forces are applied at any location along the length of the tube. However, such catheters are typically sufficiently flexible such that during deployment they can bend or curve under external forces imposed by, e.g., an operating physician at the proximal end of the catheter; by contacting an inner wall of an endoscope channel; by contacting inner walls of organs along the GI tract; or by passing the catheter over a guidewire.

Accordingly, the simple catheter can assume many variations of three-dimensional shapes due to its flexibility and various external forces imposed at various locations along its length allowing the catheter to extend along a tortuous path—e.g., extending along a tortuous path defined by a natural body lumen through which it extends. However, those skilled in the art understand that the ability of the catheter to curve or bend is generally limited and such bending typically proceeds in a snake-like manner, e.g., as body lumens are traversed. Those skilled in the art will ascertain that a simple catheter generally does not expand in shape, for example, radially, nor have additional features for expansion relative to its circular cross-section along its length.

In various exemplary embodiments described herein, a catheter device can include one or more balloons fixed to a catheter that can be introduced to the GI tract in a deflated state and, when the balloon(s) are positioned at a desired location in the GI tract, fluid can be applied to the inner volume of the balloon(s) to expand the balloon(s) into an inflated state. In some embodiments, the balloon is sized to engage the inner walls of the small bowel to anchor the balloon in place and/or expand the small bowel. In some embodiments, the balloon is filled with fluid that can provide a target for a user performing an EUS-GE procedure while, in other embodiments, the fluid providing the target is discharged proximally or distally to the balloon. In some embodiments, the balloon is designed to be punctured, e.g., by a piercing element employed in the EUS-GE procedure while, in other embodiments, the balloon can remain inflated until a desired time at which the user deflates the balloon. In various embodiments, the balloon can be filled with saline; contrast; or a fluoroscopy-visible or EUS-visible medium.

In one aspect of these exemplary embodiments, a catheter device includes a balloon located adjacent to a distal end of a catheter and three channels or lumens extending through the catheter in communication with the proximal end of the catheter. A first channel, e.g., a main lumen, is the guidewire lumen and extends fully through the catheter to its open distal end, permitting the catheter to travel over the guidewire to a target location; a second channel extends to a proximal end of the balloon and permits the anchoring balloon to be filled with saline/contrast; and a third channel communicates with exit port(s) proximal to the balloon, permitting saline/contrast to be discharged proximal to the balloon which, when the balloon is inflated, prevents the fluid from traveling distally. The physician now has a target that will not puncture when the needle is introduced proximal to the balloon as well as an anchoring balloon to hold the catheter in place. Thus, the catheter is anchored in place and the balloon prevents flow of the saline/contrast distally from the target site, providing a durable target for access.

FIG. 3 shows a catheter device 200 including a catheter 202 with a balloon 208 fixed thereto in an expanded shape (second shape) within a target portion of the anatomy 120 of FIG. 1b according to a first example of these exemplary embodiments. Similar to the NJ tube devices 100 and 152 of the preceding examples, the catheter device 200 can be introduced into the esophagus, passed through the stomach past the stricture and placed such that a distal end 204 of the catheter 202 is within the small bowel. The anatomy 120 shown in FIG. 3 comprises the jejunum 136, similar to the preceding figures.

The catheter 202 extends from a proximal end (not shown) to a distal end 204 which, in this embodiment includes an atraumatic distal tip. In this example, the distal end 204 is open to a first channel 209 (e.g., a main lumen) of the catheter 202 such that a guidewire (not shown) can be inserted within the catheter 202. The guidewire can be advanced to the jejunum 136 and the catheter 202 can be passed over the guidewire to place the distal end 204 of the catheter 202 in the jejunum 136.

In this example, the catheter device 200 further includes the balloon 208. A first distal portion (not labeled) of the catheter 202 provides a surface over which the balloon 208 can be disposed. A proximal end (not labeled) of the balloon 208 is open to a second channel/lumen (not shown) for inflating the balloon 208 with fluid (e.g., saline/contrast), to be described in greater detail below. A second distal portion 206 of the catheter 202 includes multiple holes (not labeled) for providing an outlet for fluid introduced via a third channel/lumen (not shown) of the catheter 202. In other examples, the second distal portion 206 can comprise a single hole. In this example, the second distal portion 206 is proximal to the first distal portion and the balloon 208.

The balloon 208 of this embodiment is fixed to the first distal portion of the catheter 202 at its proximal and distal ends. Prior to inflation, the balloon 208 remains in a deflated state (first shape) suitable for advancing the catheter 202 to the jejunum 136. In this example, to transition the balloon 208 into an inflated state (second shape), fluid is provided via the second channel to fill the inner volume of the balloon 208. The balloon 208 is sized and shaped so that, when inflated, the balloon 208 engages the inner walls of the jejunum 136 to expand the jejunum 136 and to anchor the catheter 202 in place within the small bowel. After anchoring the catheter 202 in place, fluid is provided via the third channel and discharged via the holes in the second distal portion 206 of the catheter 202 while the inflated balloon 208 prevents this fluid exiting the holes in the second distal portion 206 from migrating distally past the balloon 208.

In this example, the catheter device 200 is configured for use in assisting the EUS-GE procedure and the fluid discharged via the holes of the second distal portion 206 comprises, for example, contrast fluid for enhancing visualization of the small bowel. As would be understood by those skilled in the art, the catheter device 200 is positioned so that fluid is provided to a target location under visualization and so that a piercing element may be deployed from an endoscope to penetrate the stomach at a desired location to pass out of the stomach and to further penetrate the jejunum 136 at the desired location so that a gastro-jejunal stent (AXIOS stent) can be placed to bridge the desired locations. It is noted that either one or both of the fluids for inflating the balloon 208 and/or for filling the jejunum 136 proximal to the balloon can comprise saline or contrast.

It is further noted that the catheter device 200 can be modified for use as a feeding tube, e.g., by providing the second distal portion 206 with one or more exit ports distally of the balloon 208 and/or by discharging fluid directly out the first channel 209 (main lumen) after removing the guidewire (i.e., supplying fluid to the first channel 209 to pass this fluid to the exit ports. However, to utilize the anchoring properties of the catheter device 200, the balloon 208 may need to remain inflated for the duration of use as a feeding tube, which may not be feasible for extended use.

At a suitable time during the EUS-GE procedure, or upon completion of the procedure, the balloon 208 may be deflated by removing the fluid from its inner volume to disengage the balloon 208 from the inner walls of the jejunum 136 permitting the subsequent removal of the catheter device 200.

A catheter device according to an alternative embodiment may include only two lumens with a main lumen including an open distal end configured to both receive the guidewire for advancing the catheter device over the guidewire and, after withdrawing the guidewire while the catheter device remains in place, the same lumen may be used to instill saline/contrast. A second lumen can be used to inflate the balloon prior to instilling the fluid via the main lumen. In this case, the fluid would be permitted to travel the length of the main lumen and exit the open distal end to reach the target anatomical structure.

However, if a sufficient number of exit ports of sufficient size are provided proximally to the balloon, an adequate amount of fluid may exit the holes to provide visualization of a suitable target proximal to the balloon. In still another alternative, the distal end may be closed and the catheter device can be guided to the target location under alternative visualization techniques that do not employ a guidewire. In this case, only two lumens are sufficient, e.g., one for inflating the balloon and another for instilling contrast via exit ports proximal to the balloon.

In another aspect of these exemplary embodiments, a catheter device includes a first catheter (outer catheter) with a first balloon located adjacent to its distal end and a second catheter (inner catheter) with a second balloon located adjacent to its distal end. The first balloon and second balloon can be expanded, e.g., successively, to provide two anchor locations within the GI tract. A first channel/lumen of the first catheter extends fully through the first catheter to its open distal end, permitting the second catheter to be advanced through the first channel such that a distal end of the second catheter is located a desired distance distally of the distal end of the first catheter. In one exemplary method, the first catheter is advanced past the stricture so that the first balloon can be inflated on the distal side of the stricture and the second catheter is advanced further distally into the GI tract beyond the distal end of the first catheter and inflated within the jejunum. The catheter device can further comprise a ratcheting mechanism to lock the distance between the first and second balloons once these have been positioned as desired.

FIG. 4a shows a catheter device 210 including a first catheter 212 (outer catheter) with a first balloon 222 fixed thereto, a second catheter 226 with a second balloon 234 fixed thereto and a ratcheting mechanism for locking a desired distance between the balloons 222, 234 according to a second example of these exemplary embodiments; FIG. 4b shows the catheter device 210 of FIG. 4a with the second catheter 226 advanced distally relative to the first catheter 212 according to the second exemplary embodiment; FIG. 4c shows the ratcheting mechanism of the catheter device 210 of FIG. 4a according to the second exemplary embodiment; FIG. 4d shows the catheter device 210 within the anatomy 120 of FIG. 1b in a first stage of deployment according to the second exemplary embodiment; FIG. 4e shows the catheter device 210 within the anatomy 120 of FIG. 1b in a second stage of deployment according to the second exemplary embodiment. The anatomy 120 shown in FIGS. 4d-e includes the stomach 126, the pyloric stricture 138 and the small bowel 130 including the duodenum 132 and the jejunum 136.

The first catheter 212 extends from a proximal end 214 to a distal end 216 with an atraumatic distal tip that is open to a first channel 220 (e.g., a main lumen) of the first catheter 212 such that the second catheter 226 can extend through the first channel 220 and translate longitudinally relative thereto. In this example, the distal end 216 includes a number of protrusions 218 (e.g., at least two) or one unitary ring extending radially inward into the first channel 220 at or near the distal tip where the protrusions 218 form one aspect of the ratcheting mechanism as will be described in further detail below. The catheter device 210 further includes the first balloon 222 disposed around a distal portion (not labeled) of the first catheter 212 adjacent to the distal end 216. A proximal end (not labeled) of the first balloon 222 is open to a second channel/lumen (not labeled) for inflating the first balloon 222 with fluid (e.g., saline) via a port 224.

The first balloon 222 of this embodiment is fixed to the distal portion of the first catheter 212 at its proximal and distal ends. Prior to inflation, the first balloon 222 remains in a deflated state (first shape) suitable for advancing the distal end 216 of the first catheter 212 past the stricture 138 in a first stage of deployment of the catheter device 210, to be described in greater detail below. In this example, to transition the first balloon 222 into an inflated state (second shape), fluid is provided via the second channel to fill the inner volume of the first balloon 222.

The second catheter 226 extends from a proximal end 228 to a distal end 230 with an atraumatic distal tip that can be open to a third channel (e.g., guidewire lumen) (not labeled) of the second catheter 226 such that the second catheter 226 can be passed over a guidewire to the target location. In other embodiments, the distal end 230 of the second catheter 226 can be closed and the catheter device 210 can be advanced to the target location under alternative visualization methods that do not employ a guidewire.

In this example, the second catheter 226 includes a medial portion 232 comprising ridges along its length with the ridges constituting another aspect of the ratcheting mechanism as will be described in further detail below. The catheter device 210 further includes the second balloon 234 disposed around a distal portion (not labeled) of the second catheter 226 adjacent to the distal end 230. A proximal end (not labeled) of the second balloon 234 is open to a fourth channel/lumen (not labeled) for inflating the second balloon 234 with fluid (e.g., saline) via a port 236.

The second balloon 234 of this embodiment is fixed to the distal portion of the second catheter 226 at its proximal and distal ends. Prior to inflation, the second balloon 234 generally remains in a deflated state (first shape) suitable for advancing the distal end 230 of the second catheter 226 through the small bowel 130 to a desired location within, e.g., the jejunum 136, in a second stage of deployment of the catheter device 210, to be described in greater detail below. In this example, to transition the second balloon 234 into an inflated state (second shape), fluid is provided via the fourth channel to fill the inner volume of the second balloon 234.

The ratcheting mechanism of the present embodiment comprises the protrusions 218 at the distal end 216 of the first catheter 212 which are configured to engage the ridges of the medial portion 232 of the second catheter 226. The distal end 216 of the first catheter 212 and the medial portion 232 of the second catheter 226 are configured (e.g., sized, shaped and positioned) so that the ends of the protrusions 218 can be received between adjacent ridges, to form a (temporary) lock between the first and second catheters 212, 226. The protrusions 218 are sufficiently flexible such that a small force applied distally to the second catheter 226, relative to the first catheter 212 held in fixed position, deforms the protrusions 218 to allow distal translation of the second catheter 226 relative to the first catheter 212 such that the protrusions 218 are received between a more proximal pair of adjacent ridges. The second catheter 226 can therefore advance distally through the first channel 220 of the first catheter 212 and be temporarily locked in any of a plurality of positions defined by each pair of longitudinally adjacent ones of the protrusions 218. It should be understood a similar method can be used to withdraw the second catheter 226 proximally as the protrusions 218 are deformed in a proximal direction.

As shown in FIG. 4a, the second catheter 226 can extend a first distance out the distal end 216 of the first catheter 212. In this position, the protrusions 218 engage ridges located near a distal end of the medial portion 232. As shown in FIG. 4b, the second catheter 226 can extend a second distance out the distal end 216 of the first catheter 212. In this position, the protrusions 218 engage ridges located near a proximal end of the medial portion 232. As shown in FIG. 4c, the protrusions 218 are received between adjacent ridges to form a temporary lock to maintain a desired distance by which the second catheter 226 (and the second balloon 234 attached thereto) extends distally out of the distal end 216 of the first catheter 212 (and the first balloon 222 attached thereto).

As shown in FIG. 4d, the catheter device 210 is advanced through the stomach 126 and the stricture 138 with the second catheter 226 (inner catheter) extending just past the distal end 216 of the first catheter 212 (outer catheter) such that the second balloon 234 is adjacent to the first balloon 222. When the first balloon 222 (outer balloon or proximal balloon) reaches a desired location (e.g., when it has been moved distally past the stricture 138), the first balloon 222 is inflated to provide a first anchor point in the duodenum 132 adjacent to the stricture 138. The inflated first balloon 222 is sized so that it cannot pass proximally past the stricture 138.

As shown in FIG. 4e, with the first balloon 222 anchored immediately distal of the stricture 138, the second catheter 226 is advanced through the first channel 220 of the first catheter 212 to move distally relative to the first catheter 212 and distally through the small bowel 130 to a target location. The medial portion 232 of the second catheter 226 comprising the ridges engages the protrusions 218 of the first catheter 212 to provide a lock between the first catheter 212 and the second catheter 226 so that, when the second catheter 226 reaches a desired position relative to the first catheter 212, the position of the second catheter 226 is temporarily locked in the desired position by the ratchet mechanism. The second balloon 234 is then inflated to provide a second anchor point in the jejunum 136, e.g., near the ligament of Trietz.

With the balloons 222, 234 inflated at their proximal and distal positions, fluid can be instilled into an annular space within the small bowel surrounding the distal end of the first catheter 212 and the extended portion of the second catheter 226 between the balloons 222, 234. In the example shown in FIGS. 4a-b, the first catheter 212 includes an injection lumen passing through the first channel 220 (main lumen) accessible by a further port 238 so that the fluid can be instilled on the distal side of the first balloon 222. In other embodiments, the injection lumen can be in the second catheter 226 to release fluid on the proximal side of the second balloon.

In this example, the catheter device 210 is configured to assist with the EUS-GE procedure and the fluid discharged between the balloons 222, 234 comprises, in this example, contrast fluid to enhance visualization of the small bowel 130. The fluid can be provided such that a target location is provided under visualization and a piercing element deployed from an endoscope can be guided to penetrate the stomach at a desired location and to further penetrate the jejunum 136 at the desired location so that a gastro-jejunal stent (AXIOS stent) can be placed to bridge the desired locations. It is noted that the fluids for inflating the first balloon 222, inflating the second balloon 234, and/or discharge in the jejunum 136 can in various embodiments comprise either saline or contrast.

At a suitable time during the EUS-GE procedure, or upon completion, the balloons 222, 234 can be deflated by removing the fluid from their inner volumes to disengage the balloons 222, 234 from their anchor positions for subsequent removal of the catheter device 210.

In another aspect of these exemplary embodiments, a catheter device includes a first balloon (inner balloon) inside a second balloon (outer balloon) fixed to a catheter. In some embodiments, the outer balloon can be made of a material that is intended to be punctured and the inner balloon can be made of a more resilient material. In some embodiments, the inner balloon is filled with a liquid selected to aid in position confirmation, e.g., a fluoro-visible medium or an EUS-visible medium. When a target location is reached, the outer balloon can be inflated with a fluid (e.g., saline) to expand the outer balloon into contact with the walls of the small bowel to anchor the device in the small bowel at the target location.

FIG. 5 shows a catheter device 240 including an outer balloon 248 and an inner balloon 246 fixed near a distal end 244 of a catheter 242 within the anatomy 120 of FIG. 1b according to a third example of these exemplary embodiments. The anatomy 120 shown in FIG. 5 includes the jejunum 136. As described above, the inner balloon 246 is inflated to provide visualization to assist a user in positioning the balloons 246, 248 at the target location to provide visual targeting information for a procedure. When the target location is reached, the outer balloon 248 is inflated to expand the outer balloon 248 into contact with the wall of the jejunum anchoring the catheter device 240 in place.

Once this system is setup, the AXIOS device may be placed using the visible inner balloon as a target. In some embodiments, the user is intended to puncture through the outer balloon which will additionally confirm to the user that they have punctured through to the correct portion of the anatomy, e.g., the jejunum 136, which would greatly reduce AXIOS mis-deployments.

In another aspect of these exemplary embodiments, a catheter device includes a balloon with a “rolling balloon” construction wherein the balloon is fixed to a catheter at a centrally located point of the balloon. The balloon is configured so that, once inflated, the position of the balloon may be shifted relative to the attachment point. In practice, a balloon engaging an inner wall of the small bowel may be pulled out of position due to axial forces imparted by normal motion of the anatomy and/or friction with an endoscope. In these embodiments, the rolling balloon allows a user to move the device distally and proximally along the jejunum to counteract such movements. When the axial forces are removed, the balloon would then return to the neutral position at which it was initially placed.

The balloon can be attached to the catheter at a small central location on the balloon. The balloon is configured so that, once inflated, the balloon is able to shift position relative to the attachment point. The radially outer surface of the balloon could also be constructed with ribs or other methods to allow the balloon to ‘stick’ to the inside of the jejunum.

FIG. 6a shows a catheter device 250 including a rolling balloon 260 fixed to a catheter 252 within the patient anatomy of FIG. 1b according to a fourth example of these exemplary embodiments; FIG. 6b shows the balloon 260 of the catheter device 250 of FIG. 6a displaced proximally in the patient anatomy of FIG. 1b according to the fourth exemplary embodiment; FIG. 6c shows the balloon 260 of the catheter device 250 of FIG. 6a displaced distally in the patient anatomy of FIG. 1b according to the fourth exemplary embodiment. The anatomy 120 shown in FIGS. 6a-c includes the jejunum 136.

The catheter 252 extends from a proximal end (not shown) to a distal end 254 with an atraumatic distal tip. A first distal portion 256 of the catheter 252 provides a surface over which the balloon 260 can be attached. In this example, the balloon 260 is attached to the catheter 252 at only the first distal portion 256, e.g., at a single location, such that the balloon 260 can displace proximally and/or distally under proximal/distal forces as the volume within the balloon 260 is displaced and the balloon 260 deforms and/or rolls longitudinally along the catheter 252. As shown in FIG. 6a, in this example, the balloon 260 is attached at a central location 262 on the balloon 260 when the balloon 260 is at rest. The first distal portion 256 may also communicate with a first channel or lumen for inflating/deflating the balloon 260. A second distal portion 258 of the catheter 252 includes multiple holes (not labeled) for providing an outlet for fluid introduced via a second channel/lumen (not shown) of the catheter 252. In other examples, the second distal portion 258 can comprise a single hole. In this example, the second distal portion 258 is proximal to the first distal portion 256 and the balloon 260.

Prior to inflation, the balloon 260 is maintained in a deflated state (first shape) suitable for advancing the catheter 252 to the jejunum 136. In this example, to transition the balloon 260 into an inflated state (second shape), fluid is provided via the first channel to fill the inner volume of the balloon 260. The balloon 260 is configured (e.g., sized and shaped) so that, when inflated, it will engage the inner walls of the jejunum 136 to anchor the catheter 252 in place within the small bowel. After anchoring the catheter 252 in place, fluid can be provided via the second channel and discharged via the holes in the second distal portion 258 of the catheter 252. The inflated balloon 260 prevents the fluid from migrating distally.

The location 262 on the balloon 260 where the balloon 260 attaches to the catheter 252 permits the balloon 260 to displace proximally and distally relative to the catheter 252 under forces imposed by the anatomy 120 during use. If, during deployment, the balloon 260 encounters axial forces, e.g., proximal or distal forces, the balloon 260 can roll along the inner wall of the jejunum 136, as shown in FIGS. 6b-c.

In this example, the catheter device 250 is configured for assisting the EUS-GE procedure and the fluid discharged via the holes of the second distal portion 258 can comprise contrast fluid for visualizing the small bowel. The fluid can be provided such that a target location is provided under visualization and a piercing element deployed from an endoscope can penetrate the stomach at a desired location and further penetrate the jejunum 136 at the desired location so that a gastro-jejunal stent (AXIOS stent) can be placed to bridge the desired locations. It is noted that either one or both of the fluids for inflating the balloon 260 and/or for filling the jejunum 136 proximal to the balloon can comprise saline or contrast.

It is further noted that the catheter device 250 can be modified for use as a feeding tube, e.g., by providing the second distal portion 258 with the exit ports distally to the balloon 260 and/or by discharging fluid directly out a main lumen. However, the balloon 260 would need to remain inflated for the duration of use as a feeding tube, which may not be feasible for extended use.

In another aspect of these exemplary embodiments, micropatterning can be added to the surface of a balloon to enhance adhesion between the balloon and the jejunum wall. Micropatterns such as micro-scale pillars have been studied extensively as effective tissue adhesives. Micropatterning one or more of the balloons of the preceding embodiments allows the balloon surfaces to be more adhesive through a combination of force (interlock) as the balloon expands and suction, increasing its ability to adhere to the walls of the jejunum.

As would be understood by those skilled in the art, the micropattern may be applied directly into the balloon (such that the balloon and the micropattern are the same material) or the micropattern may be applied to the balloon after its manufacture (allowing the micropattern to be a different material than the balloon). The micropattern may be applied throughout the surface of the balloon, or on specific segments of the balloon surface. A level of desired adhesion balanced with cost and manufacturing constraints may dictate micropatterning only part of the balloon surface. Additionally, oils and other mucoadhesive substances could be applied to the portions of the balloons containing micropattern prior to use to enhance the effectiveness/optimize the function of the micropattern.

FIG. 7a shows a EUS-GE system 270 comprising a balloon 274 with a first micropatterned surface 276 according to a fifth example of these exemplary embodiments. In this example, the EUS-GE system 270 is similar to the EUS-GE system 150 of FIG. 2 and includes a catheter 272 on which the balloon 274 is fixed. The first micropatterned surface 276 comprises substantially rectangular protrusions.

FIG. 7b shows a second micropatterned surface 278 according to a sixth example of these exemplary embodiments. The second micropatterned surface 278 comprises protrusions that flair radially.

FIG. 7c shows a third micropatterned surface 280 according to a seventh example of these exemplary embodiments. The third micropatterned surface 280 comprises protrusions that are rounded, e.g., in an egg-like shape.

FIG. 7d shows a fourth micropatterned surface 282 according to an eighth example of these exemplary embodiments. The fourth micropatterned surface 282 comprises protrusions with a mushroom top.

It should be understood that the micropatterned surface can be used in combination with a number of the preceding embodiments, including, e.g., the balloons 208, 222, 234, 248 and 260.

It may be noted by those knowledgeable in the art that any of the above embodiments may be combined in any manner not inconsistent with their operation and design to provide a system to enable ostomy management utilizing any or all of the characteristics of the various embodiments.

1-15. (canceled)

16. A device for administering a fluid to a patient anatomy, comprising:
a catheter including a first channel extending therethrough for passing the catheter over a guidewire, the catheter being sized and shaped to extend from a proximal end outside a human body to a distal end that can be guided to a target location within a gastrointestinal (GI) system, a first distal portion of the catheter including one or more exit ports communicating with a second channel for administering a fluid to the target location; and
a balloon distal to the exit ports, the catheter including a third channel extending therethrough communicating with an interior of the balloon so that, in a first state, the balloon is deflated and assumes a first shape suitable for guiding the catheter to the target location and, in a second state, the balloon is inflated and assumes a second shape in which the balloon extends radially from the catheter for contacting a wall of the target location to anchor the catheter in the target location while administering the fluid via the first channel.

17. The device of claim 16, wherein the fluid administered to the target location comprises contrast and the balloon prevents the fluid from migrating distally.

18. The device of claim 16, wherein the balloon comprises a micropatterned surface for adhering to the wall of the target location.

19. A device for administering a fluid to a patient anatomy, comprising:
a first catheter that is an outer catheter including a first channel extending therethrough, the first catheter being sized and shaped to extend from a proximal end outside a human body to a distal end that can be guided to a first target location within a gastrointestinal (GI) system;
a first balloon adjacent to the distal end of the first catheter, the first catheter including a second channel extending therethrough communicating with an interior of the first balloon so that, in a first state, the first balloon is deflated and assumes a first shape suitable for guiding the first catheter to the first target location and, in a second state, the first balloon is inflated and assumes a second shape in which the first balloon extends radially from the first catheter for contacting a wall of the first target location to anchor the first catheter in the first target location;
a second catheter that is an inner catheter sized and shaped to be received within the first channel of the first catheter, the second catheter extending from a proximal end outside the human body through the first channel to a distal end that that, in a first position, is distal to the distal end of the first catheter, the second catheter being translatable relative to the first catheter such that, after the first balloon has anchored the first catheter in the first target location, the second catheter can be guided to a second target location within the GI system; and
a second balloon adjacent to the distal end of the second catheter, the second catheter including a third channel extending therethrough communicating with an interior of the second balloon so that, in a first state, the second balloon is deflated and assumes a first shape suitable for guiding the second catheter to the second target location and, in a second state, the second balloon is inflated and assumes a second shape in which the second balloon extends radially from the second catheter for contacting a wall of the second target location to anchor the second catheter in the second target location.

20. The device of claim 19, further comprising a ratcheting mechanism wherein a medial portion of the second catheter comprises ridges along a length of the medial portion, wherein the distal end of the first catheter comprises a protrusion extending radially inward to engage a space between adjacent ridges to maintain a position of the second catheter relative to the first catheter, the protrusion being flexible so that an axial force applied to the second catheter deforms the protrusion allowing the second catheter to translate so that the protrusion engages a further space between further adjacent ridges on the medial portion.

21. The device of claim 19, wherein either the first catheter or the second catheter includes a fourth channel for administering a fluid to a volume between the first and second target locations, the volume being distal to the first balloon and proximal to the second balloon.

22. The device of claim 19, wherein either one or both of the first balloon and the second balloon comprises a micropatterned surface for adhering to the wall of the first target location or the second target location.

23. A device for administering a fluid to a patient anatomy, comprising:
a catheter sized and shaped to extend from a proximal end outside a human body to a distal end that can be guided to a target location within a gastrointestinal (GI) system;
a first balloon that is an inner balloon adjacent to the distal end of the catheter, the catheter including a first channel extending therethrough communicating with an interior of the first balloon so that, in a first state, the first balloon is deflated and assumes a first shape suitable for guiding the catheter to the target location and, in a second state, the first balloon is inflated with a fluid having properties to enhance visibility for a fluoroscopic or endoscopic procedure; and
a second balloon that is an outer balloon disposed around the first balloon, the catheter including a second channel extending therethrough communicating with an interior of the second balloon so that, in a first state, the second balloon is deflated and assumes a first shape suitable for guiding the catheter to the target location and, in a second state, the second balloon is inflated and assumes a second shape in which the second balloon extends radially from the catheter for contacting a wall of the target location to anchor the catheter in the target location.

24. The device of claim 23, wherein the first balloon provides a target for the fluoroscopic or endoscopic procedure and is formed of a first material stronger than a second material of the second balloon.

25. The device of claim 24, wherein the second balloon is punctured and the first balloon is not punctured during the fluoroscopic or endoscopic procedure.

26. The device of claim 25, wherein the second balloon comprises a micropatterned surface for adhering to the wall of the target location.

27-35. (canceled)
